(19) United States
(12) Patent Application Publication
(10) Pub. No.: US 2019/0060181 A1
(43) Pub. Date: Feb. 28, 2019

(54) Skin Care Facial Mask Stick and Methods Of Use Thereof

(71) Applicant: Elica Lemann, Las Vegas, NV (US)

(72) Inventor: Elica Lemann, Las Vegas, NV (US)

(21) Appl. No.: 15/684,874

(22) Filed: Aug. 23, 2017

(51) Int. Cl. A61K 8/02; A61K 8/73; A61K 8/9789; A61K 8/97; A61Q 19/00; A61Q 19/08; A45D 40/20

(52) U.S. Cl. CPC A61K 8/0229; A61K 8/0212; A61K 8/73; A61K 8/9789; A61K 8/97; A61Q 19/00; A45D 2040/208; A45D 40/205; A61K 2800/48; A61K 2800/524; A61K 2800/85; A61K 2800/87; A61Q 19/08

(57) ABSTRACT

The present invention relates to improving the appearance of a person, particularly the facial skin, and to improve health and damage of cells and tissues preferably facial skin, and more preferably providing the skin with vitamins, healthy antioxidants. Embodiments of the present invention overcome deficiencies in the art by providing a skin care facial mask stick containing raw mask material that can be used in skin treatment applications.

### FIELD OF THE INVENTION

The present invention relates in general to the field of skin treatment. More particularly, the present invention relates to a skin care facial mask stick and methods of use thereof.

### BACKGROUND OF THE INVENTION

With aging, chronic exposure to adverse environmental factors, or malnutrition, the visual appearance and physical properties of skin can change in ways that are considered visually undesirable. The most notable and obvious changes include the development of fine lines and wrinkles, loss of elasticity, increased sagging, loss of firmness, loss of color evenness or tone, coarse surface texture, and mottled pigmentation. People can also develop skin conditions such as acne, a condition that develops when hair follicles plug with oil and dead skin cells.

Current facial masks intended to address this problem are typically provided in liquid or paste form in a tube. These products are applied by either using one's hands or by dipping a brush into the product and spreading the product on the skin by the brush. These existing products can cause contamination and bacterial spread from the user's hands or the brush to be unintentionally applied to the user's face. Additionally, these products are not convenient to use. For example, a user may scoop too much of the product via a brush or their hands than would be necessary, therefor creating additional mess and hassle and wasting more mask material than necessary. A user may spill the product out of a tube, or drop it due to the additional complexity of use.

Furthermore, current facial masks are comprised of oils which are heavy and therefore take a long time to be absorbed by the skin. The average wait time for a current facial mask is between ten to thirty minutes long. The masks have been designed to stay on the skin for a long time, and may contain additional harmful chemicals. Many of these masks also use preservatives such as Phenoxyethanol which can cause harm to human body.

Therefore, there is a strong need in the art for a facial mask that is more hygienic and convenient to use in order to minimize the spread of bacteria and encourage higher user compliance rates. There is also a strong need to shorten the wait time to use the mask. It is to these ends and overcoming the other issues presented in existing products that the present invention has been developed.

### SUMMARY

The present invention relates to improving the appearance of a person, particularly the facial skin, and to improve health and damage of cells and tissues preferably facial skin, and more preferably providing the skin with vitamins, healthy antioxidants. Embodiments of the present invention overcome deficiencies in the art by providing a skin care facial mask stick containing raw mask material that can be used in skin treatment applications.

A person can more conveniently put a facial mask on via a facial mask stick in order to promote brighter, smoother, vitamin rich, and healthier looking skin. Embodiments of the present invention overcome deficiencies in the art by providing a skin care facial mask stick containing raw mask material that can be used in skin treatment applications.

The raw mask material of the present invention may include vegetable or fruit fruit puree and/or juice. The raw mask material may also include naturally derived preservatives such as Radish Root Ferment Filtrate (Leucidal Liquid). In other non-limiting aspects of the present invention, the raw mask material can further include a vitamin, a mineral, an essential fatty acid, an amino acid, a flavonoid, and/or a protein, or a combination thereof. Non-limiting examples of vitamins include the B vitamins (e.g., B1, B2, B6, B12, niacin, folic acid, biotin, and pantothenic acid), vitamin C, vitamin D, vitamin E (e.g., tocopherol or tocopheryl acetate), vitamin A (e.g., palmitate, retinyl palmitate, or retinoic acid), and vitamin K. Non-limiting examples of minerals include iron, potassium, phosphorus, magnesium, manganese, selenium, and calcium. Non-limiting examples of essential fatty acids include Omega 3 (linolenic acid), Omega 6 (linoleic acid) and Omega 9 (oleic acid) essential fatty acid, or a combination thereof. Non-limiting examples of amino acids include essential amino acids (e.g., lysine, leucine, isoleucine, methionine, phenylalanine, threonine, tryptophan, valine, histidine, or arginine) and non-essential amino acids (e.g., serine, asparagine, glutamine, aspartic acid, glutamic acid, alanine, tyrosine, cysteine, glycine, or proline). Non-limiting examples of flavonoids include anthocyanin compounds (e.g., cyanidin-3-glucoside and cyanidin-3-rutinoside).

The mask may also include organic lycium barbarum (whole wolfberry) puree, organic vaccinium corymbosum (blueberries), organic rubus idaeus (raspberry) puree, cranberry puree, macha green tea extract, glycerin (vegetable derived), prunus avium (sweet cherry) fruit extract, fragaria ananassa (strawberry) fruit extract, 1-ascrobic acid (vitamin c), bambusa vulgaris (bamboo) extract, accinium macrocarpon (cranberry) seeds, ubiquinone (co-q-10), alkanet root extract, organic rubus idaeus (raspberry) puree, macha green tea, organic punica granatum (pomegranate), glycerin (vegetable derived), organic prunus ameniaca (apricot) puree, organic vitis vinifera (grape) seed, 1-ascrobic acid (vitamin c), glycerin (vegetable drived), bambusa vulgaris (bamboo) extract, sunflower oil, cetearyl olivate, sorbitan olivate, accinium macrocarpon (cranberry) seeds, ubiquinone (co-q-10), organic citrus limonum (lemon) rind, organic citrus sinensis (orange) rind, spinach puree, kale puree, montmorillonite (french green) clay, montmorillonite (bentonite) clay, aloe barbadensis (aloe vera) gel, glycerin (vegetable derived), aqua (ph-balanced distilled water), salicylic acid, spirulina maxima, laminaria digitata (seaweed) extract, matcha green tea extract, radish root ferment filtrate (leucidal liquid).

In a particular embodiment there is disclosed a method of applying a skin care facial mask on a user's face, the method comprising:

providing a solid mask stick, the solid mask stick comprising a mask material housed in a tubular dispenser;
turning a screw mechanism on the bottom of the tubular dispenser for the mask material to protrude out of the tubular dispenser;

applying the solid mask stick to the face without touching the mask material.

It can be appreciated that by utilizing a solid mask stick, the facial mask can be applied directly to a user's face without the user's hands touching the product. In a further embodiment, the mask material is suitable for all skin types even the most sensitive.

In a further embodiment, a raw material is combined with a thickening agent as part of the process of forming the mask material into a solid mask stick. In a further embodiment, the thickening agent is Xanthan Gum.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented below.

FIG. 1 discloses a side view of a facial mask stick

FIG. 2 discloses a method of applying a skin care facial mask stick.

### DETAILED DESCRIPTION

In today's image conscious society, people are continually looking for a face mask product that can improve the visual appearance of their skin. Often times, aged skin, uneven skin tone, or skin damaged by environmental factors such as UV light, chronic sun exposure, environmental pollutants, chemicals, disease pathologies, or smoking, is associated with unattractive skin. Previous face masks has been shown to have various drawbacks as described above.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as claimed.

The present invention relates to improving the appearance of a person, particularly the facial skin, and to improve health and damage of cells and tissues preferably facial skin, and more preferably restoring aged facial skin to a youthful appearance. Embodiments of the present invention overcome deficiencies in the art by providing a skin care facial mask stick containing raw mask material that can be used in skin treatment applications.

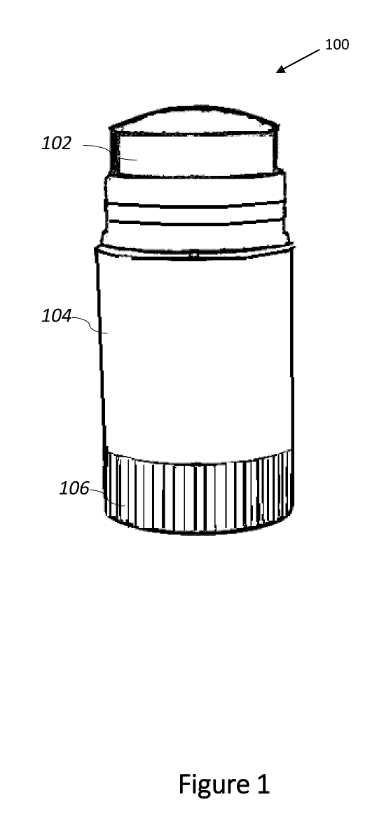

FIG. 1 discloses a mask stick 100 in accordance with an embodiment of the invention. A solid mask stick 102 is housed in a tubular dispenser 104. The mask stick can be moved upwards out of the tubular dispenser 104 by turning the turn portion 106 of the tubular dispenser. In one embodiment, the solid mask stick is composed of a raw mask material that has been mixed with a thickening agent.

Further embodiments of the present invention disclose creating a raw mask material for treating the skin on the face or the body, and forming the material into a solid mask stick which is housed in a tubular dispenser. In an embodiment, the raw mask material is combined with a thickening agent as part of the process of forming the material into a solid mask stick. In a further embodiment, the thickening agent is Xantham Gum.

In one embodiment, the raw material is comprised of fruit and vegetables purees along with juices. It can be appreciated that because the concentration of vitamins in such a raw mask material is very high, the mask would only require a two to three minute wait time as opposed to a much longer wait time for existing facial masks. It can further be appreciated that because the fruit and vegetable purees and juices allow the active ingredients (the combination of fruit and vegetable juices and purees) to travel through the skin layers faster, there is provided a faster absorption rate.

In other non-limiting aspects of the present invention, the raw mask material can further include a vitamin, a mineral, an essential fatty acid, an amino acid, a flavonoid, and/or a protein, or a combination thereof. Non-limiting examples of vitamins include the B vitamins (e.g., B1, B2, B6, B12, niacin, folic acid, biotin, and pantothenic acid), vitamin C, vitamin D, vitamin E (e.g., tocopherol or tocopheryl acetate), vitamin A (e.g., palmitate, retinyl palmitate, or retinoic acid), and vitamin K. Non-limiting examples of minerals include iron, potassium, phosphorus, magnesium, manganese, selenium, and calcium. Non-limiting examples of essential fatty acids include Omega 3 (linolenic acid), Omega 6 (linoleic acid) and Omega 9 (oleic acid) essential fatty acid, or a combination thereof. Non-limiting examples of amino acids include essential amino acids (e.g., lysine, leucine, isoleucine, methionine, phenylalanine, threonine, tryptophan, valine, histidine, or arginine) and non-essential amino acids (e.g., serine, asparagine, glutamine, aspartic acid, glutamic acid, alanine, tyrosine, cysteine, glycine, or proline). Non-limiting examples of flavonoids include anthocyanin compounds (e.g., cyanidin-3-glucoside and cyanidin-3-rutinoside).

The mask may also include organic lycium barbarum (whole wolfberry) puree, organic vaccinium corymbosum (blueberries), organic rubus idaeus (raspberry) puree, cranberry puree, macha green tea extract, glycerin (vegetable derived), prunus avium (sweet cherry) fruit extract, fragaria ananassa (strawberry) fruit extract, 1-ascrobic acid (vitamin c), bambusa vulgaris (bamboo) extract, accinium macrocarpon (cranberry) seeds, ubiquinone (co-q-10), alkanet root extract, organic rubus idaeus (raspberry) puree, macha green tea, organic punica granatum (pomegranate), glycerin (vegetable derived), organic prunus ameniaca (apricot) puree, organic vitis vinifera (grape) seed, 1-ascrobic acid (vitamin c), glycerin (vegetable drived), bambusa vulgaris (bamboo) extract, sunflower oil, cetearyl olivate, sorbitan olivate, accinium macrocarpon (cranberry) seeds, ubiquinone (co-q-10), organic citrus limonum (lemon) rind, organic citrus sinensis (orange) rind, spinach puree, kale puree, montmorillonite (french green) clay, montmorillonite (bentonite) clay, aloe barbadensis (aloe vera) gel, glycerin (vegetable derived), aqua (ph-balanced distilled water), salicylic acid, spirulina maxima, laminaria digitata (seaweed) extract, matcha green tea extract, radish root ferment filtrate (leucidal liquid)

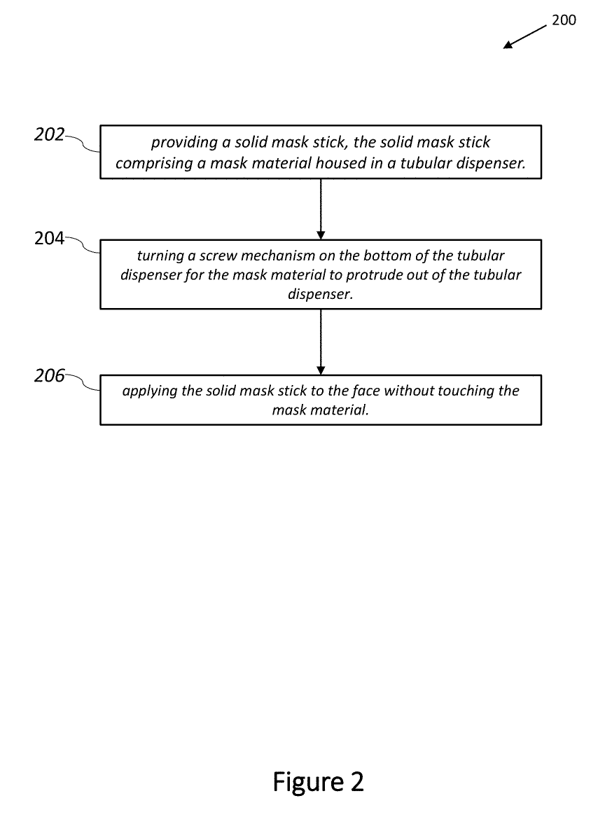

FIG. 2 discloses a particular embodiment a method 200 of applying a skin care facial mask on a user's face. Step 202 discloses providing a solid mask stick, the solid mask stick comprising a mask material housed in a tubular dispenser. Step 204 discloses turning a screw mechanism on the bottom of the tubular dispenser for the mask material to protrude out of the tubular dispenser. Step 206 discloses applying the solid mask stick to the face without touching the mask material.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method of applying a skin care facial mask on a user's face, the method comprising:
providing a solid mask stick, the solid mask stick comprising a mask material housed in a tubular dispenser;
turning a screw mechanism on the bottom of the tubular dispenser for the mask material to protrude out of the tubular dispenser;
applying the mask material to the face without touching the mask material.

2. The method of claim 1, wherein the mask material comprises a raw material combined with a thickening agent.

3. The method of claim 2, wherein the thickening agent is xanthan gum.

4. The method of claim 3, wherein the raw material comprises any combination of fruit juices, vegetable juices, fruit purees, or vegetable purees.

5. The method of claim 4, wherein the raw material further comprises a natural preservative.

6. The method of claim 5, wherein the preservative is Radish Root Ferment Filtrate.

7. A facial mask stick, comprising:
a mask material housed in a tubular dispenser;
a screw mechanism on the bottom of the tubular dispenser, wherein turning the screw mechanism causes the mask material to protrude out of the tubular dispenser.

8. The facial mask stick of claim 7, wherein the mask material comprises a raw material combined with a thickening agent.

9. The facial mask stick of claim 8, wherein the thickening agent is xanthan gum.

10. The facial mask stick of claim 9, wherein the raw material comprises any combination of fruit juices, vegetable juices, fruit purees, or vegetable purees.

11. The facial mask stick of claim 10, wherein the raw material further comprises a natural preservative.

12. The facial mask stick of claim 11, wherein the preservative is Radish Root Ferment Filtrate.
